(19) United States
(12) Patent Application Publication
(10) Pub. No.: US 2015/0328250 A1
(43) Pub. Date: Nov. 19, 2015

(54) Animal Food Composition and Method

(71) Applicant: HILL'S PET NUTRITION, INC., Topeka, KS (US)

(72) Inventors: Luis J. MONTELONGO, Lawrence, KS (US); Pavinee CHINACHOTI, Muang (TH)

(21) Appl. No.: 14/652,777

(22) PCT Filed: Dec. 19, 2012
(86) PCT No.: PCT/US2012/070511

§ 371 Date: Jun. 16, 2015

(51) Int. Cl. A61K 31/722; A23K 1/18; A23K 1/175

(52) U.S. Cl. CPC A61K 31/722; A23K 1/1758; A23K 1/1853

(57) ABSTRACT

The present invention provides an animal food composition comprising a food component, chitosan and a benefit agent, wherein the benefit agent forms a complex with the chitosan and wherein the chitosan-benefit agent complex forms a gel at a pH in the range from about 7 to about 8. Methods for delivery of a benefit agent and for treating an oral cavity condition wherein the benefit agent is a therapeutic agent are disclosed.

### BACKGROUND OF THE INVENTION

Animals, including companion animals such as felines and canines require oral care. Poor oral health can cause animals pain and serious dental problems throughout life, as well as possibly leading to more serious illnesses such as heart and kidney disease. Veterinary dental specialists have previously estimated that 70% of cats and 80% of dogs have some form of gum disease by the age of 3. Unhealthy gums, including gingival inflammation or gingivitis are considered to be a common oral health issue affecting companion animals.

Oral care products are available for animals but providing proper oral care remains a challenge. For example, regular brushing by the animal owner, though beneficial, can be an inconvenient chore that is difficult to perform or carry out on a regular basis. Conventional hard, crunchy dry foods, chew toys and the like fail to remove plaque and tartar at the gum line and are inadequate to promote periodontal health. Nutritional solutions have been proposed, therefore. For example, ingestion of edible compositions which comprise oral health promoting components is one approach.

In a further approach, used largely for human oral hygiene certain polymer compositions have been proposed in oral care compositions, including the use of chitosan. Chitosan is a deacetylated form of chitin, a naturally-occurring substance principally derived from the cuticles of crustacea. Chitin has a chemical structure that consists of 2-aceytamido-2-deoxy-beta-D-glucose monomers attached via a beta-1-4 linkage. Chitosan is fairly unusual in that it is a cationic polysaccharide, bearing a positive charge due to the presence of protonated amino groups along its backbone. As a consequence, it is soluble at low pH only.

US2006/0134011 describes an oral and dental composition for use as an antibacterial toothpaste. The composition comprises a combination of divalent ions, the quaternary ammonium compound cetylpyridinium chloride and encapsulated or derivatized chitosan. These types of chitosan are soluble at oral cavity pH, which is approximately neutral pH.

There remains a need to provide for effective delivery of agents to the oral cavity, particularly to promote oral health in animals.

### BRIEF SUMMARY OF THE INVENTION

In a first aspect, the present invention provides an animal food composition comprising a food component, chitosan and a benefit agent, wherein the benefit agent forms a complex with the chitosan and wherein the chitosan-benefit agent complex forms a gel at a pH in the range from about 7 to about 8.

In a second aspect, the present invention provides such a composition for use in the treatment of an oral cavity condition, wherein the benefit agent comprises a therapeutic agent.

In a third aspect, the present invention provides a method for treating an oral cavity condition in an animal, which comprises feeding the animal with a composition as described herein, wherein the benefit agent comprises a therapeutic agent.

In a fourth aspect, the present invention provides a method for delivering a benefit agent to the oral cavity of an animal, which comprises feeding the animal an animal food composition comprising a food component, chitosan and the benefit agent, wherein the benefit agent forms a complex with the chitosan and wherein the chitosan-benefit agent complex forms a gel at a pH in the range from about 7 to about 8 and wherein the gel adheres to the teeth or gums to provide for delivery of the benefit agent.

It has surprisingly been found that compositions according to the present invention contain a complex between the benefit agent and the chitosan which, at oral cavity pH provides for sustained release of the benefit agent into the oral cavity. When feeding the chitosan complexed with the benefit agent, the chitosan-benefit agent complex lodges around the teeth and gums of the animal, and the chitosan in the gel is slowly broken down thereby releasing the benefit agent from the gel. This has applicability in the maintenance of oral health and in the treatment of oral cavity conditions and may also provide for sustained-release into the GI tract. Prior art arrangements using encapsulated or derivatized chitosan are not gel-forming at oral cavity pH.

### DETAILED DESCRIPTION OF THE INVENTION

The following description of the preferred embodiment(s) is merely exemplary in nature and is in no way intended to limit the invention, its application, or uses.

As used herein, the term “food” may refer not only to a food product which typically provides most, if not all, the nutrient value for an animal, but may also refer to such items as a snack, treat, and supplement.

According to the invention, an animal food composition comprises a food component, chitosan and a benefit agent. The benefit agent forms a complex with the chitosan. The chitosan-benefit agent complex forms a gel at a pH in the range from about 7 to about 8.

The term “benefit agent” as used in the context of the present invention denotes any agent which improves oral health or provides oral care.

The benefit agent may be selected from a metal ion, a nutrient, an antimicrobial, a nutraceutical, a probiotic, a prebiotic or a mixture of two or more of these agents. Chitosan is capable of forming a complex with a wide range of benefit agents. In one arrangement, the benefit agent is ionic and may comprise a cation such as a metal ion. In one arrangement, the benefit agent comprises a divalent metal ion such as Zn²⁺, Cu²⁺Fe²⁺. Metal ions such as these possess antimicrobial properties. Zinc ions are particularly useful because, in addition to their antimicrobial properties, zinc can fight plaque, prevent calculus formation and reduce mouth malodor. Zinc may also be used in the treatment and prevention of other oral conditions such as pyorrhea and tooth hypersensitivity.

In one arrangement, the divalent metal ion is provided as a water-soluble zinc salt such as zinc acetate, zinc chloride or zinc sulfate.

The zinc salts may form part of an antimicrobial system which includes other antimicrobial compounds. Because the present invention provides for sustained release of the benefit agent, where the benefit agent is a zinc ion it is possible to use the zinc ion alone in the antimicrobial system. In one arrangement, therefore, the antimicrobial system consists essentially of a water-soluble zinc salt as the benefit agent which forms a complex with the chitosan. The composition according to the invention may therefore be free from other antimicrobial compounds such as quaternary ammonium compounds. Some such compounds possess poor organoleptic properties and may have an unpleasant taste.

The composition may comprise zinc ions at a concentration in the range of from 50 ppm to 1000 ppm, by total weight of the composition. Preferably, the composition comprises zinc ions at a concentration in the range of from 100 ppm to 1000 ppm by total weight of the composition, or from 100 ppm to 500 ppm by total weight of the composition. In some embodiments, the composition may comprise zinc ions at a concentration in the range of from 100 ppm, or 200 ppm, or 300 ppm, or 400 ppm, to 500 ppm, or 600 ppm, or 700 ppm, or 800 ppm, or 900 ppm, or 1000 ppm by total weight of the composition.

Chitosan is a cationic copolymer comprising glucosamine and N-acetylglucosamine monomeric units, with a predominance of glucosamine monomeric units. The chitosan may be provided in the present invention in the form of a salt such as chitosan lactate or chitosan acetate.

The chitosan used in the invention forms a gel at a pH in the range of from about 7 to about 8. Typically, the gel adheres to the teeth or gums to provide for delivery of the benefit agent. Gel formation may therefore occur at one or more pHs in that range. It is not necessary for gel formation to occur at all pHs in the range provided that a gel is formed at the pH of the oral cavity of the animal for whom the food composition is intended. Preferably, the chitosan forms a gel at a pH in the range of from about 7.3 to about 7.8. This is the typical pH range for dog saliva. The average value for cat saliva is pH 7.5. Gel formation is indicated by the detection of any precipitate in a chitosan containing composition. This is typically determined by separating precipitate from a liquid phase using a filtration system such as Whatman filter paper 40.

Grades of chitosan which have the properties according to the invention may be obtained from standard sources including “tasteless/odourless” chitosan, high deacetylation chitosan or chitoclear chitosan. Chitosan salts such as chitosan lactate are another grade of chitosan which may be used according to the invention. Grades which are not suitable include those which are soluble at around neutral pH such as encapsulated chitosan and N-derivatized chitosan such as N-carboxybutyl chitosan and N-carboxymethyl chitosan.

The chitosan may be present in the composition in an amount of 0.01 wt % to 3 wt % by total weight of the composition. In one embodiment, the chitosan is present in the composition in an amount of 0.05 wt. % to 2 wt % by total weight of the composition. Preferably, the chitosan is present in the composition at a concentration in the range of from 0.1 wt % to about 1 wt % by total weight of the composition. More preferably, chitosan is present in the composition at a concentration in the range of from 0.1 wt % or 0.2 wt % or 0.3 wt %, or 0.4 wt %, or 0.5 wt %, or 0.6 wt %, or 0.7 wt %, or 0.8 wt % or 0.9 wt % by total weight of the composition.

The food composition of the present invention is for animal consumption. The term “animal” in the context of the present invention includes human and non-human animals. Non-human animals include, without limitation, avians, bovines, canines, equines, felines, murines, ovines, and porcines. Preferably, the food composition is for consumption by a companion animal such as a canine or a feline.

The food component may include, without limitation, protein, grains, cereal flour, starch, fats, oils, vitamins, minerals, colorants, flavorants, amino acids, fiber and inorganic additives. The compositions of the present invention may comprise one or more of these ingredients.

Protein may be derived from an animal source or a vegetable source. Animal protein sources include meat, meat by-products and fish. Meat sources may comprise animal muscle, animal skeletal meat, animal by-products, and mixtures of muscle, skeletal meat and byproducts. Meats include, for example, the flesh of poultry and mammals (e.g. chickens, cattle, swine, sheep, goats, and the like). Meat by-products include, for example, lungs, kidneys, livers, tongues, stomachs and intestines. Suitable animal protein sources may include fresh and frozen meats or meat by-products.

Vegetable protein sources suitable for preparing compositions of the invention include, but are not limited to, soy concentrate, soy protein isolate, soybean meal, corn gluten meal, rice protein isolate, pea protein concentrate, wheat protein concentrate, and wheat protein isolate. Vegetable protein may be isolated from any portion of a plant, isolated from more than one portion of a plant, and isolated from more than one plant by methods known by those of skill in the art. Vegetable protein may also be concentrated by methods known by those of skill in the art.

Protein may be present in the food composition in an amount of from 15 wt % to 35 wt %, by total weight of the food composition. Preferably, protein is present in the food composition in an amount of about from 20 wt % to 30 wt %, or from 20 to 25 wt %, or from 20 to 22 wt % by total weight of the food composition.

Suitable grains for use in the compositions of the present invention may be selected from the group consisting of corn, sorghum, wheat, rye, barley, oat and mixtures thereof. It is important to note that by properly balancing carbohydrate sources, one skilled in the art can manipulate the texture of the final product. For example, short chain polysaccharides tend to be more ‘sticky’ and ‘gluey’ than longer chain polysaccharides.

The food composition may comprise grains in an amount of from 30 wt % to 50 wt % by total weight of the food composition. Preferably, grains are present in the food composition in an amount of from 35 to 50 wt %, or from 40 wt % to 50 wt %, or from 45 to 50 wt % by total weight of the food composition.

Vitamins generally useful in the compositions of the present invention include, for example, vitamin A, vitamin B1, vitamin B2, vitamin B6, vitamin B12, vitamin C, vitamin D, vitamin E, vitamin H (biotin), vitamin K, folic acid, inositol, niacin, and pantothenic acid. Minerals and trace elements generally useful as food additives in the food composition include, for example, calcium, phosphorus, sodium, potassium, magnesium, copper, choline, and iron salts.

The food composition may comprise vitamins in an amount of from 0.05 wt % to 5 wt % by total weight of the food composition. Preferably, vitamins are present in the food composition in an amount of about from 0.1 to 3 wt %, or from 0.1 wt % to 2 wt %, or from 0.1 to 1 wt % by total weight of the food composition.

The food composition may comprise minerals and trace elements in an amount of from 0.05 wt % to 5 wt % by total weight of the food composition. Preferably, minerals and trace elements are present in the food composition in an amount of about from 0.1 to about 3 wt %, or from 0.1 wt % to 2 wt %, or from 0.1 to 1 wt % by total weight of the food composition.

Fat can be supplied by any of a variety of sources known by those skilled in the art, including chicken fat, beef tallow, choice white grease, vegetable oils and mixtures thereof.

The food composition may comprise fat in an amount of from 2 wt % to 30 wt %, by total weight of the food composition. Preferably, fat present in the food composition in an amount of from 5 wt % to 20 wt %, or from 7 wt % to 17 wt %, or from 10 to 15 wt % by total weight of the food composition.

Amino acids, including essential amino acids, free amino acids, or amino acids supplied by any number of sources, e.g., crude protein, may be present in the food composition of the present invention. Essential amino acids are amino acids that cannot be synthesized de novo, or in sufficient quantities by an organism and thus must be supplied in the diet. Essential amino acids vary from species to species, depending upon the organism's metabolism. For example, it is generally understood that the essential amino acids for dogs and cats (and humans) are phenylalanine, leucine, methionine, lysine, isoleucine, valine, threonine, tryptophan and histidine. In addition, taurine, which is technically not an amino acid but a derivative of cysteine, is an essential nutrient for cats.

The food composition may comprise amino acids in an amount of from 0.05 wt % to 5 wt % by total weight of the food composition. Preferably, amino acids are present in the food composition in an amount of about from 0.1 to 3 wt %, or from 0.1 wt % to 2 wt %, or from 0.1 to 1 wt % by total weight of the food composition.

The food composition may additionally comprise a source of dietary fiber. Dietary fiber refers to components of a plant which are resistant to digestion by an animal's digestive enzymes. Dietary fiber includes soluble and insoluble fibers. Soluble fibers are resistant to digestion and absorption in the small intestine and undergo complete or partial fermentation in the large intestine. Soluble fibers include beet pulp, guar gum, chicory root, psyllium, pectin, blueberry, cranberry, squash, apples, oats, beans, citrus, barley, or peas. Insoluble fibers may be supplied by any of a variety of sources, including cellulose, whole wheat products, wheat oat, corn bran, flax seed, grapes, celery, green beans, cauliflower, potato skins, fruit skins, vegetable skins, peanut hulls, and soy fiber.

The food composition may comprise fiber in an amount of from about 1 wt % to 20 wt % by total weight of the food composition. Preferably, fiber present in the food composition in an amount of about from 5 wt. % to 20 wt %, or from 7 wt % to 17 wt %, or from 10 to 15 wt % by total weight of the food composition.

The food composition may additionally comprise stabilizers, fillers, thickeners, and palatability enhancers.

The amount of the optional ingredients defined herein for incorporation into the compositions of the present invention is at least partially dependent on the nutritional requirements for animals at different stages of life. The Association of American Feed Control Officials (AAFCO), for example, provides recommended amounts of such ingredients for dogs and cats. (See Association of American Feed Control Officials, Inc., Official publication, pp. 147-153 (2011)).

Typically, the food composition is frangible in the oral cavity of the animal, and preferably, the food composition is chewable. By chewing the food composition, the chitosan complexed with the benefit agent (in gel form) is advantageously delivered all around the oral cavity, and becomes lodged in the teeth. The chitosan may subsequently be gradually broken down (e.g., through salivary action or water), thereby releasing the benefit agent into the oral cavity.

The food composition of the present invention may be prepared in a dry using conventional processes. Preferably, the food composition is a dry food composition which may be in the form of a kibble.

In one embodiment, a dry food composition is prepared by grinding and mixing dry ingredients, including animal protein sources, plant protein sources, and grains. Moist or liquid ingredients, including fats, oils, animal protein sources, and water, are then added to and blended with the dry mix. The resulting mixture may then be processed into kibbles using extrusion and cutting processes known to the person skilled in the art.

The chitosan and benefit agent may be added to the food composition during its normal preparation procedure such as mixing, extrusion, and baking. The chitosan and benefit agent may be added simultaneously or sequentially. Preferably, they are added after the food composition has been prepared (for example, post extrusion), in the form of a solution.

Typically, the chitosan and benefit agent are present in a coating on the food composition. In one arrangement, the chitosan and benefit agent are simultaneously applied to the food composition, in solution, to coat the food composition. Thus, the chitosan and benefit agent may be added in a single solution. In a preferred arrangement, the food composition is sprayed with, or dipped in, a solution comprising the chitosan and benefit agent to form the coated food composition.

In another arrangement, the chitosan and benefit agent may be applied to the food composition in a sequential manner. Thus, two separate solutions comprising chitosan and the benefit agent, respectively, may be applied to the food composition.

In solution, chitosan forms a complex with benefit agents such as zinc, copper and iron ions. A chitosan solution for application to the food composition preferably has a pH of less than 6 such that the chitosan remains soluble until it is brought into contact with the oral cavity of an animal, wherein the higher oral cavity pH results in gel formation. This provides for a sustained release of the benefit agent into the oral cavity.

The food composition may further be coated with an edible oil such as soybean oil, rapeseed oil, coconut oil and palm oil to improve the palatability of the food composition. In a preferred arrangement, the food composition is coated with the edible oil prior to coating with the chitosan and benefit agent. Preferably, the edible oil is soybean oil.

The food composition may comprise edible oil in an amount of from 0.5 wt % to 10 wt % by total weight of the food composition. Preferably, the food composition comprises edible oil in an amount of from 1 wt % to 7 wt %, or from 2 wt % to 5 wt %, or from 3 wt % to 4 wt %, by total weight of the food composition.

In one arrangement, the food composition of the present invention is provided as a nutritionally complete food composition. A “nutritionally complete composition” is a composition that includes sufficient nutrients for maintenance of normal health of a healthy animal consuming the composition.

In another arrangement, the food composition of the present invention may be in the form of a treat, snack, or supplement. Treats of the present invention can be prepared by extrusion or baking processes that would be apparent to the person skilled in the art. A solution comprising the chitosan and benefit agent may be used to either coat the exterior of an existing treat form, or it may be injected into an existing treat form.

The present invention further provides a use of the compositions of the present invention as defined above for treating a condition of the oral cavity. Conditions of the oral cavity that may be treated include without limitation, pyorrhea, tooth hypersensitivity, oral malodor, tooth decay, periodontal disease, plaque and gingivitis. Accordingly, the benefit agent may comprise a therapeutic agent. Therapeutic agents useful for treating disease conditions of the oral cavity would be well known to the person skilled in the art.

Accordingly, the present invention provides a composition as defined above for use as a medicament.

The present invention further provides a method for delivering a benefit agent to the oral cavity of an animal, which comprises feeding the animal an animal food composition comprising a food component, chitosan and a benefit agent, wherein the benefit agent forms a complex with the chitosan, wherein the chitosan-benefit agent complex forms a gel at a pH in the range from about 7 to about 8, and wherein the gel adheres to the teeth or gums to provide for delivery of the benefit agent.

Typically, the gel containing the complex of the chitosan and the benefit agent forms in the oral cavity of the animal. Chewing stimulates saliva production, which in turn raises the pH throughout the oral cavity to a range of from about 7 to 8, thereby resulting in gel formation.

The formed gel lodges around the teeth and gums of the animal consuming the composition. As the chitosan is broken down (e.g. by salivary action or water), the benefit agent is gradually released into the oral cavity. Accordingly, the delivery of the benefit agent may be a sustained-release delivery.

The present inventors have surprisingly found that compositions according to the present invention contain a complex between the benefit agent and the chitosan which, at oral cavity pH provides for sustained release of the benefit agent into the oral cavity. When feeding the chitosan complexed with the benefit agent, the chitosan-benefit agent complex lodges around the teeth and gums of the animal, and the chitosan in the gel is slowly broken down thereby releasing the benefit agent from the gel. The chitosan gel is subsequently slowly broken down thereby releasing the benefit agent from the gel. This has applicability in the maintenance of oral health and in the treatment of oral cavity conditions and may also provide for sustained-release into the gastro-intestinal tract.

The invention is further illustrated in the following non-limiting Examples.

### Example 1

### Sustained Release of Zinc

The chitosan-controlled release of zinc was investigated. 0.1% solutions of chitosan were prepared. Zinc acetate was added to each of these solutions to provide a zinc ion concentration of 100 ppm or 200 ppm. The pH of the solutions was adjusted to 2, 5 or 7. Each solution was left at room temperature for 72 hours while gently mixing to allow for complete chitosan dissolution. Solutions containing zinc alone acted as controls for the experiment.

Each solution was subsequently transferred to dialysis tubing and placed in a jar containing deionized water. Stirring was carried out continuously throughout the duration of dialysis. Evaporative loss was minimized by placing a sealing wrap over the jar. A sample of the dialysate was removed at regular intervals and the zinc ion concentration was measured by inductively coupled plasma optical emission spectrometry (ICP-OES).

Table 1 illustrates the amount of zinc ions released from the 100 ppm solutions at pH 2, 5 and 7 over time. Table 2 illustrates the amount of zinc ions released from the 200 ppm solutions at pH 2, 5 and 7 over time.

TABLE 1

| | pH 7 | | pH 5 | | pH 2 | |
|---|---|---|---|---|---|---|
| | Sample | | | | | |
| Time | Chit/Zinc | Zinc | Chit/Zinc | Zinc | Chit/Zinc | Zinc |
| (hrs) | Amount of Zn++ (mg) | | | | | |
| 0.00 | 0 | 0 | 0 | 0 | 0 | 0 |
| 0.33 | 0.10 ± 0.00 | 0.30 ± 0.00 | 0.50 ± 0.00 | 0.57 ± 0.01 | 0.40 ± 0.00 | 0.25 ± 0.00 |
| 0.67 | 0.20 ± 0.00 | 0.71 ± 0.15 | 0.98 ± 0.00 | 1.15 ± 0.02 | 0.79 ± 0.00 | 0.42 ± 0.01 |
| 1.00 | 0.29 ± 0.00 | 0.82 ± 0.00 | 1.46 ± 0.00 | 1.68 ± 0.02 | 1.17 ± 0.00 | 0.52 ± 0.01 |
| 1.33 | 0.44 ± 0.05 | 1.11 ± 0.00 | 1.98 ± 0.05 | 2.36 ± 0.07 | 1.65 ± 0.00 | 0.75 ± 0.01 |
| 1.67 | 0.58 ± 0.00 | 1.40 ± 0.01 | 2.21 ± 0.00 | 2.86 ± 0.07 | 1.82 ± 0.00 | 0.90 ± 0.02 |
| 2.00 | 0.71 ± 0.05 | 1.78 ± 0.25 | 2.57 ± 0.00 | 3.14 ± 0.01 | 2.14 ± 0.05 | 1.01 ± 0.02 |
| 2.50 | 0.85 ± 0.00 | N/A | 2.92 ± 0.00 | 3.69 ± 0.05 | 2.50 ± 0.05 | 1.40 ± 0.01 |
| 3.00 | 1.03 ± 0.00 | N/A | 3.23 ± 0.05 | 4.00 ± 0.00 | 2.71 ± 0.00 | 1.66 ± 0.02 |

TABLE 2

| | pH 7 | | pH 5 | | pH 2 | |
|---|---|---|---|---|---|---|
| Time | Chit/Zinc | Zinc | Chit/Zinc | Zinc | Chit/Zinc | Zinc |
| (hrs) | Amount of Zn++ (mg) | | | | | |
| 0.00 | 0 | 0 | 0 | 0 | 0 | 0 |
| 0.33 | 0.15 ± 0.05 | 0.630 ± 0.00 | 0.94 ± 0.05 | 0.99 ± 0.00 | 0.99 ± 0.10 | 0.52 ± 0.00 |
| 0.67 | 0.34 ± 0.05 | 1.21 ± 0.01 | 1.77 ± 0.00 | 1.91 ± 0.02 | 1.77 ± 0.00 | 1.07 ± 0.00 |
| 1.00 | 0.49 ± 0.00 | 1.76 ± 0.01 | 2.59 ± 0.05 | 2.73 ± 0.00 | 2.59 ± 0.05 | 1.77 ± 0.07 |
| 1.33 | 0.68 ± 0.00 | 2.36 ± 0.02 | 3.44 ± 0.05 | 3.61 ± 0.00 | 3.29 ± 0.00 | 2.36 ± 0.01 |
| 1.67 | 0.86 ± 0.00 | 2.88 ± 0.02 | 3.94 ± 0.00 | 4.25 ± 0.01 | 3.84 ± 0.00 | 2.90 ± 0.01 |
| 2.00 | 1.10 ± 0.05 | 3.25 ± 0.04 | 4.62 ± 0.05 | 4.85 ± 0.02 | 4.47 ± 0.00 | 3.40 ± 0.05 |
| 2.50 | 1.23 ± 0.00 | N/A | 5.38 ± 0.09 | 5.59 ± 0.01 | 5.05 ± 0.05 | 4.09 ± 0.01 |
| 3.00 | 1.22 ± 0.00 | N/A | 5.80 ± 0.00 | 6.13 ± 0.03 | 5.66 ± 0.05 | 4.59 ± 0.00 |

Table 3 illustrates the average rate of zinc release per hour from both the 100 ppm and 200 ppm solutions, in the presence and absence of chitosan.

TABLE 3

| | |
|---|---|
| 100 ppm Zn acetate | 200 ppm Zn acetate |

| | | | |
|---|---|---|---|
| Zinc | Chit/Zinc | Zinc | Chit/Zinc |

| | |
|---|---|
| pH | mg/hr |

| | | | | |
|---|---|---|---|---|
| 2 | 0.54 | 0.93 | 1.58 | 1.88 |
| 5 | 1.38 | 1.09 | 2.07 | 1.97 |
| 7 | 0.85 | 0.35 | 1.47 | 0.45 |

As can be seen from Tables 1, 2 and 3, the presence of chitosan significantly reduces the rate of zinc release at pH 7, but not at pH 2 or pH 5. At pH 2, chitosan is highly soluble and repulsion between the protonated chitosan and positive zinc ions promotes release of the zinc ions. Accordingly, a higher rate of zinc release may be seen in the presence of chitosan than in the absence of chitosan. At pH 5, a small difference in the rate of zinc release is observed between the solutions containing chitosan and the solutions containing no chitosan. No gel formation is observed at pH 5. At pH 7, the presence of chitosan significantly reduces the rate of zinc release and gel formation is observed which shows chitosan forming a complex with zinc ions resulting in a reduced rate of release of the zinc.

The amount of zinc bound to chitosan after 1 hour of dialysis at pH 2, 5 and 7 was also determined After 1 hour of dialysis, the amount of zinc that had been released through the dialysis tubing, in the presence and absence of chitosan, was measured as described above. The amount of zinc released in the presence of chitosan was subtracted from the amount of zinc released in the absence of chitosan to determine the amount of zinc bound to chitosan. The results are indicated in Table 4.

TABLE 4

| Variable | Zn++ | Zn++ | Zinc bound | Bound Zinc |
|---|---|---|---|---|
| Zinc⁺⁺ (ppm) | no chitosan | chitosan | (ppm) | per gram of |
| solution at | (A) | (B) | (A − B) | chitosan |
| different pH's | (mg) | (mg) | (mg) | (mg) |
| 100 ppm pH 2 | 0.57 | 1.10 | −0.54 | −5.36 |
| 200 ppm pH 2 | 1.68 | 2.34 | −0.66 | −6.61 |
| 100 ppm pH 5 | 1.62 | 1.34 | 0.28 | 2.86 |
| 200 ppm pH 5 | 2.54 | 2.40 | 0.14 | 1.38 |
| 100 ppm pH 7 | 0.87 | 0.37 | 0.50 | 4.98 |
| 200 ppm pH 7 | 1.68 | 0.50 | 1.19 | 11.86 |

As can be seen from Table 4, and consistent with the zinc release rates discussed above, the amount of bound zinc per gram chitosan is greatest at pH 7. (The negative values for bound zinc per gram of chitosan observed at pH 2 may be attributed to the increased zinc release in the presence of chitosan relative to the zinc release observed in the absence of chitosan, as described above).

These results support the use of chitosan in the sustained delivery of benefit agents in an oral cavity to improve oral health.

### Example 2

### The Effect of pH on the Gelling Properties of Chitosan

A 1% chitosan solution was prepared by mixing 1 g of chitosan with 75 ml of an acidic solution and stirring for 15 minutes. Additional acidic solution was then added to make the final volume up to 100 ml, and the solution was mixed for a further 2 hours and 45 minutes prior to storing at 4° C. Three different chitosan sources were used: “tasteless/odourless” chitosan, high deacetylation chitosan and chitoclear chitosan.

The pH of 10 g samples of the solution was adjusted to 2, 3, 4, 5, 5.5, 5.75, 6, 7 or 8 using 1 or 0.1 N NaOH, or 1 or 0.1N HCl. The samples were then centrifuged at 3200 rpm for 20 minutes at room temperature to allow gel (precipitate) formation.

The centrifuged samples were then filtered through a pre-weighed (weight 1), oven-heated and desiccated Whatman filter paper 40. Any gel formed was retained by the filter. After filtering, the filter papers (with any collected gel) was placed in an oven overnight, prior to weighing again (weight 2). Weight 1 was subtracted from weight 2 to quantify the amount of gel formation. Assays were conducted in triplicate.

The results are illustrated in Table 5.

TABLE 5
Tasteless/Odorless

| pH | Sample 1 | 2 | 3 | Average |
|---|---|---|---|---|
| 2.00 | 0.009 | 0.003 | 0.000 | 0.004 |
| 3.00 | 0.009 | 0.002 | 0.002 | 0.004 |
| 4.00 | 0.002 | 0.005 | 0.006 | 0.004 |
| 5.00 | 0.015 | 0.006 | 0.002 | 0.007 |
| 5.50 | 0.010 | 0.006 | 0.008 | 0.008 |
| 5.75 | 0.009 | 0.005 | 0.013 | 0.009 |
| 6.00 | 0.024 | 0.015 | 0.002 | 0.014 |
| 7.00 | 0.100 | 0.086 | 0.082 | 0.089 |
| 8.00 | 0.086 | 0.067 | 0.123 | 0.092 |

High Deacetylation

| pH | B1 | B2 | B3 | Average |
|---|---|---|---|---|
| 2.00 | 0.006 | 0.002 | −0.006 | 0.001 |
| 3.00 | 0.012 | 0.011 | 0.010 | 0.011 |
| 4.00 | 0.011 | 0.012 | 0.001 | 0.008 |
| 5.00 | 0.012 | 0.020 | 0.021 | 0.018 |
| 5.50 | 0.028 | 0.046 | 0.014 | 0.029 |
| 5.75 | 0.055 | 0.053 | 0.054 | 0.054 |
| 6.00 | 0.058 | 0.059 | 0.042 | 0.053 |
| 7.00 | 0.108 | 0.100 | 0.093 | 0.100 |
| 8.00 | 0.099 | 0.093 | 0.075 | 0.089 |

Chitoclear

| pH | D1 | D2 | D3 | Average |
|---|---|---|---|---|
| 2.00 | 0.007 | 0.003 | 0.009 | 0.0065 |
| 3.00 | −0.006 | 0.004 | 0.002 | 0.0000 |
| 4.00 | 0.005 | 0.012 | 0.010 | 0.0089 |
| 5.00 | 0.020 | 0.037 | 0.013 | 0.0232 |
| 5.50 | 0.013 | 0.029 | 0.038 | 0.0266 |
| 5.75 | 0.034 | 0.045 | 0.028 | 0.0357 |
| 6.00 | 0.059 | 0.058 | 0.061 | 0.0593 |
| 7.00 | 0.109 | 0.099 | 0.097 | 0.1018 |
| 8.00 | 0.098 | 0.102 | 0.093 | 0.0977 |

It can be seen from Table 5 that all of the chitosans formed a gel (precipitate) at a pH higher than 6. Gel quantification showed that maximum gel formation occurs at pH 7 and 8, very little gel formation occurs at pH 6, and almost no gel formation occurs at lower pHs for all chitosans tested.

Due to the increased gel formation properties of chitosan at pH 7 to 8 which is comparable to the pH range of the saliva of dogs and cats, chitosan may be used for the sustained delivery of benefit agents (e.g. ions, nutrients, antimicrobials, nutraceuticals, probiotics, prebiotics, and other compounds) to the oral cavity of cats and dogs.

### Example 3

### Release of Zinc from Kibbles Coated with Chitosan and Zinc Solutions

A 2% chitosan lactate solution and a 4000 ppm zinc⁺⁺ (zinc acetate) solution were prepared. The pH of the zinc solution was 6.33 and the pH of the chitosan solution was 3.57.

Dog kibbles were added to a mixer and coated with soybean oil (such that the final concentration of soybean oil was approximately 4.4 wt % by total weight of the kibbles). The kibbles were then sprayed with the chitosan lactate solution, followed by the zinc acetate solution to achieve a final concentration of 5 wt. % of the chitosan lactate solution and 5 wt. % of the zinc acetate solution by total weight of the kibbles. (This resulted in a delivery of −0.1% chitosan and 200 ppm of zinc⁺⁺ onto the kibbles.) The kibbles were subjected to continuous mixing during the spraying.

The rate of zinc release from the kibbles was determined by mixing 30 g of the coated kibbles in 270 ml deionized water at 37° C. Samples of the water were removed at regular intervals and the amount of zinc in these samples was measured by ICP-OES as described above.

To act as a negative control for the experiment, kibbles were coated with zinc acetate solution and soybean oil as described above, in the absence of any chitosan coating.

The results are indicated in Table 6.

TABLE 6

| | Chitosan Solution & | |
|---|---|---|
| Time | Zinc Acetate Solution | Zinc Acetate Solution |
| (seconds) | Zinc (mg/L) | Zinc (mg/L) |
| 0 | 0.17 | 0.00 |
| 15 | 4.91 | 7.44 |
| 30 | 8.07 | 12.20 |
| 45 | 10.34 | 15.51 |
| 60 | 11.73 | 17.77 |
| 75 | 12.99 | 19.72 |
| 90 | 13.91 | 20.09 |
| 105 | 14.52 | 20.62 |
| 120 | 14.83 | 21.23 |

As can be seen in Table 6, the sample coated with the chitosan solution and zinc acetate solution had a significantly reduced rate of zinc release into the water, as compared to the sample coated with zinc acetate solution alone. Chitosan is a powerful chelating agent which forms complexes with the zinc ions, thereby preventing their release.

### Example 4

### Release of Zinc from Kibbles Coated with Chitosan Solution and Zinc Powder

A 1% aqueous acidic chitosan solution was prepared and sprayed onto kibbles in an amount of 10 wt %, by total weight of the kibbles. Zinc acetate powder was then applied to the kibbles in an amount of 0.264 wt % by total weight of the kibbles (to deliver ˜700 ppm zinc to the kibbles), while continuously mixing for 5 minutes.

The rate of zinc release from the kibbles was determined by mixing 20 g of the coated kibbles in 300 ml deionized water at 37° C. Samples of the water were removed at regular intervals and the amount of zinc in these samples was measured by ICP-OES as described above.

To act as a control for the experiment, an acidic solution comprising 1% chitosan and 2. 64% zinc acetate was sprayed onto kibbles in an amount of 10 wt % by total weight of the kibbles (to deliver 700 ppm zinc to the kibbles), while mixing for 5 minutes. The rate of zinc release from the kibbles was measured as above.

The results are indicated in Table 7.

TABLE 7

| | Chitosan Solution and | Chitosan and |
|---|---|---|
| Time | Zinc Acetate Powder | Zinc Acetate Solution |
| (seconds) | Zinc (mg/L) | Zinc (mg/L) |
| 0 | 0.0 | 0.0 |
| 15 | 7.3 | 2.7 |
| 30 | 10.7 | 4.0 |
| 45 | 12.7 | 5.1 |
| 60 | 13.6 | 6.0 |
| 75 | 14.2 | 6.9 |
| 90 | 15.0 | 7.7 |
| 105 | 15.7 | 8.7 |
| 120 | 16.6 | 9.6 |

As can be seen in Table 7, the sample coated with the chitosan and zinc acetate solution had a significantly reduced rate of zinc release into the water, as compared to the sample coated with chitosan solution and zinc acetate powder. These results demonstrate that zinc ions must be in solution in order to form a complex with chitosan.

### Example 5

### Food Composition According to the Present Invention

Table 8 illustrates a food composition according to the present invention.

TABLE 8

| Ingredient Name | Formula Percentage |
|---|---|
| Meat and meat by-products | 20-40 |
| Vegetable protein source | 10-30 |
| Grains | 30-50 |
| Fiber | 5-20 |
| Edible Oils | 2-5 |
| Palatability enhancer | 0.5-3 |
| Amino acids | 0.1-1 |
| Minerals and Trace elements | 0.1-1 |
| Vitamins | 0.1-1 |
| Water | q.s. |
| Total | 100.00 |

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. All percentages expressed herein are by weight of the composition on dry matter basis unless specifically stated otherwise.

What is claimed is:

1. An animal food composition comprising a food component, chitosan and a benefit agent, wherein the benefit agent forms a complex with the chitosan and wherein the chitosan-benefit agent complex forms a gel at a pH in the range from 7 to 8.

2. A composition according to claim 1, wherein the benefit agent is selected from the group comprising a metal ion, a nutrient, an antimicrobial, a nutraceutical, a probiotic, a prebiotic or a mixture thereof.

3. A composition according to claim 2, wherein the benefit agent comprises a divalent metal ion.

4. A composition according to claim 3, wherein the divalent metal ion is Zn²⁺.

5. A composition according to claim 4, wherein the divalent metal ion is provided as a water-soluble zinc salt.

6. A composition according to claim 5, wherein the water-soluble zinc salt is zinc acetate, zinc chloride or zinc sulphate.

7. A composition according to any one of claims 3 to 6 which is free from quaternary ammonium compounds.

8. A composition according to any one of the preceding claims, wherein the composition is coated with the chitosan-benefit agent complex.

9. A composition according to claim 8, wherein the benefit agent comprises zinc ions at a concentration in the range of from 100 to 1000 ppm by weight of the composition

10. A composition according to claim 8 or 9, wherein the chitosan is present at a concentration in the range of from 0.1 to 1% by weight of the composition.

11. A composition according to any one of claims 8 to 10, wherein the coating further comprises an edible oil.

12. A composition according to claim 11, wherein the edible oil comprises soybean oil.

13. A composition according to any one of claims 1 to 12 which is in the form of a kibble.

14. A composition according to any one of the preceding claims, wherein the food composition is nutritionally complete.

15. A composition according to any one of the preceding claims, wherein the animal is a companion animal.

16. A composition according to claim 15, wherein the companion animal is a canine or a feline.

17. A composition according to any one of the preceding claims for use in the treatment of an oral cavity condition, wherein the benefit agent comprises a therapeutic agent.

18. A method for treating an oral cavity condition in an animal, which comprises feeding the animal with a composition according to any one of claims 1 to 17, wherein the benefit agent comprises a therapeutic agent.

19. A method for delivering a benefit agent to the oral cavity of an animal, which comprises feeding the animal an animal food composition comprising a food component, chitosan and the benefit agent, wherein the benefit agent forms a complex with the chitosan, wherein the chitosan-benefit agent complex forms a gel at a pH in the range from about 7 to about 8, and wherein the gel adheres to the teeth or gums to provide for delivery of the benefit agent.

20. A method according to claim 19, wherein the gel containing the chitosan-benefit agent complex is formed in the oral cavity.

21. A method according to claim 19 or claim 20, wherein the delivery of the benefit agent is a sustained-release delivery.

22. A method according to any one of claims 19 to 21, wherein the benefit agent is selected from a metal ion, a nutrient, an antimicrobial, a nutraceutical, a probiotic, a prebiotic or a mixture thereof.

23. A method according to claim 22, wherein the benefit agent comprises a divalent metal ion.

24. A method according to claim 23, wherein the divalent metal ion is Zn²⁺.

25. A method according to claim 24, wherein the divalent metal ion is provided as a water-soluble zinc salt.

26. A method according to claim 25, wherein the water-soluble zinc salt is zinc acetate, zinc chloride or zinc sulphate.

27. A method according to any one of claims 23 to 26 which is free from quaternary ammonium compounds.

28. A method according to any one of claims 19 to 27, wherein the composition is coated with the chitosan-benefit agent complex.

29. A method according to claim 28, wherein the benefit agent comprises zinc ions at a concentration in the range of from 100 to 1000 ppm by weight of the composition.

30. A method according to claim 28 or 29, wherein the chitosan is present at a concentration in the range of from 0.1 to 1%, by weight of the composition.

31. A method according to any one of claims 28 to 30, wherein the coating further comprises an edible oil.

32. A method according to claim 31, wherein the edible oil comprises soybean oil.

33. A method according to any one of claims 19 to 32, wherein the composition is in the form of a kibble.

34. A method according to any one of claims 19 to 33, wherein the food composition is nutritionally complete.

35. A method according to any one of claims 19 to 34, wherein the animal is a companion animal.

36. A method according to claim 35, wherein the companion animal is a canine or a feline.
